(19) United States
(12) Patent Application Publication
(10) Pub. No.: US 2023/0381127 A1
(43) Pub. Date: Nov. 30, 2023

(54) COMPOSITIONS AND METHODS FOR MOOD ENHANCEMENT

(71) Applicant: EPITRACKER, INC., San Diego, CA (US)

(72) Inventor: Stephanie Venn-Watson, San Diego, CA (US)

(21) Appl. No.: 18/170,379

(22) Filed: Feb. 16, 2023

Related U.S. Application Data
(63) Continuation of application No. PCT/US2021/046556, filed on Aug. 18, 2021.
(60) Provisional application No. 63/068,263, filed on Aug. 20, 2020.

(51) Int. Cl. A61K 31/20; A61P 25/24; A61K 9/00

(52) U.S. Cl. CPC A61K 31/20; A61P 25/24; A61K 9/0053

(57) ABSTRACT

Compositions including odd chain saturated fatty acids, and salts and derivatives thereof, and methods for boosting and/or enhancing mood, lowering anxiety and/or pain, treating depression, treating major depressive disorder, or treating seasonal affective disorder are provided.

### INCORPORATION BY REFERENCE TO RELATED APPLICATIONS

This application is a continuation of PCT/US2021/046556 filed Aug. 18, 2021, which claims the benefit of U.S. Provisional Application No. 63/068,263, filed Aug. 20, 2020. The aforementioned application is incorporated by reference herein in its entirety; and is hereby expressly made a part of this specification.

### FIELD OF THE INVENTION

Compositions including odd chain saturated fatty acids, and salts and derivatives thereof, and methods for boosting and/or enhancing mood, lowering anxiety and/or pain, treating depression, treating major depressive disorder, or treating seasonal affective disorder are provided.

### BACKGROUND OF THE INVENTION

Mood disorders or pain increase the risk of health conditions that can decrease quality of life and longevity. Individuals suffering from mood disorders or pain are at a higher risk of developing a suite of conditions, including cardiovascular disease, fatty liver disease, proinflammatory state, and prothrombotic state. Mood disorders and pain have been identified as a causative or contributing factor to these conditions, and as such, treatment of mood disorders and pain has been proposed as a means to treat or prevent these conditions, thereby improving health and quality of life.

### SUMMARY OF THE INVENTION

Compositions and methods for boosting and/or enhancing mood, lowering anxiety and/or pain, treating depression, treating major depressive disorder, or treating seasonal affective disorder are provided. These compositions comprise one or more odd chain saturated fatty acids, derivatives of odd chain saturated fatty acids, or salts thereof, which may be administered in combination with other medicaments or supplements or as part of various treatment regimens as described herein.

Accordingly, in a generally applicable first aspect (i.e., independently combinable with any of the aspects or embodiments identified herein), a method is provided of boosting and/or enhancing mood, lowering anxiety and/or pain, treating depression, treating major depressive disorder, or treating seasonal affective disorder, comprising: administering, to a patient in need thereof, an effective amount of a C15:0 fatty acid or pharmaceutically acceptable salt thereof, in a pharmaceutical composition, a dietary supplement, or a food.

In an embodiment of the first aspect (i.e., independently combinable with any of the aspects or embodiments identified herein), the method is for boosting and/or enhancing mood.

In an embodiment of the first aspect (i.e., independently combinable with any of the aspects or embodiments identified herein), the method is for lowering anxiety.

In an embodiment of the first aspect (i.e., independently combinable with any of the aspects or embodiments identified herein), the method is for lowering pain.

In an embodiment of the first aspect (i.e., independently combinable with any of the aspects or embodiments identified herein), the method is for treating depression.

In an embodiment of the first aspect (i.e., independently combinable with any of the aspects or embodiments identified herein), the method is for treating major depressive disorder.

In an embodiment of the first aspect (i.e., independently combinable with any of the aspects or embodiments identified herein), the method is for treating seasonal affective disorder.

In an embodiment of the first aspect (i.e., independently combinable with any of the aspects or embodiments identified herein), a serum, plasma, or a red blood cell membrane concentration of the C15:0 fatty acid is increased to a concentration greater than 2.2 μM and less than 30 μM.

In an embodiment of the first aspect (i.e., independently combinable with any of the aspects or embodiments identified herein), the C15:0 fatty acid is pentadecanoic acid.

In an embodiment of the first aspect (i.e., independently combinable with any of the aspects or embodiments identified herein), the C15:0 fatty acid or pharmaceutically acceptable salt thereof is provided as a pharmaceutical composition in a unit dosage form comprising the C15:0 fatty acid or pharmaceutically acceptable salt thereof and a pharmaceutically acceptable carrier.

In an embodiment of the first aspect (i.e., independently combinable with any of the aspects or embodiments identified herein), the unit dosage form comprises from 0.01 mg to 10000 mg of the C15:0 fatty acid or pharmaceutically acceptable salt thereof.

In an embodiment of the first aspect (i.e., independently combinable with any of the aspects or embodiments identified herein), the pharmaceutical composition is substantially free from even chain saturated fatty acids.

In an embodiment of the first aspect (i.e., independently combinable with any of the aspects or embodiments identified herein), the pharmaceutical composition is substantially free from polyunsaturated fatty acids.

In an embodiment of the first aspect (i.e., independently combinable with any of the aspects or embodiments identified herein), the C15:0 fatty acid or pharmaceutically acceptable salt thereof is administered to the patient once per day.

In an embodiment of the first aspect (i.e., independently combinable with any of the aspects or embodiments identified herein), the patient is a human.

In an embodiment of the first aspect (i.e., independently combinable with any of the aspects or embodiments identified herein), the patient is a mammal.

In an embodiment of the first aspect (i.e., independently combinable with any of the aspects or embodiments identified herein), the patient is a domesticated animal.

In an embodiment of the first aspect (i.e., independently combinable with any of the aspects or embodiments identified herein), the domesticated animal is a dog or a cat.

In an embodiment of the first aspect (i.e., independently combinable with any of the aspects or embodiments identified herein), the domesticated animal is a cow, a pig, a sheep, a goat, a horse, a turkey, a duck, or a chicken.

In an embodiment of the first aspect (i.e., independently combinable with any of the aspects or embodiments identified herein), from 0.1 mg or less to 500 mg or more, e.g., 0.2 mg to 20 mg, 2.5 mg to 50 mg, e.g., 1.0 to 5.0 mg, e.g., 20 to 500 mg, e.g., 20 to 200 mg, e.g., 100 mg, of the C15:0 fatty acid or pharmaceutically acceptable salt thereof, per 1 kg of body weight, per day, is administered to the patient. In some embodiments, the effective amount of the C15:0 fatty acid or pharmaceutically acceptable salt thereof in a pharmaceutical composition is from 0.2 to 20 mg/kg body weight.

In a generally applicable second aspect (i.e., independently combinable with any of the aspects or embodiments identified herein), a composition is provided for boosting and/or enhancing mood, lowering anxiety and/or pain, treating depression, treating major depressive disorder, or treating seasonal affective disorder, comprising: C15:0 fatty acid or pharmaceutically acceptable salt thereof; and a pharmaceutically acceptable carrier.

In an embodiment of the second aspect (i.e., independently combinable with any of the aspects or embodiments identified herein), the composition is a pharmaceutical composition in unit dosage form.

In an embodiment of the second aspect (i.e., independently combinable with any of the aspects or embodiments identified herein), the composition is a dietary supplement.

In an embodiment of the second aspect (i.e., independently combinable with any of the aspects or embodiments identified herein), the dietary supplement is in unit dosage form.

In an embodiment of the second aspect (i.e., independently combinable with any of the aspects or embodiments identified herein), the dietary supplement is in a form adapted to be combined with or added to a food, beverage, or other comestible.

In an embodiment of the second aspect (i.e., independently combinable with any of the aspects or embodiments identified herein), the composition is a food or other comestible.

In an embodiment of the second aspect (i.e., independently combinable with any of the aspects or embodiments identified herein), the composition is for boosting and/or enhancing mood.

In an embodiment of the second aspect (i.e., independently combinable with any of the aspects or embodiments identified herein), the composition is for lowering anxiety.

In an embodiment of the second aspect (i.e., independently combinable with any of the aspects or embodiments identified herein), the composition is for lowering pain.

In an embodiment of the second aspect (i.e., independently combinable with any of the aspects or embodiments identified herein), the composition is for treating depression.

In an embodiment of the second aspect (i.e., independently combinable with any of the aspects or embodiments identified herein), the composition is for treating major depressive disorder.

In an embodiment of the second aspect (i.e., independently combinable with any of the aspects or embodiments identified herein), the composition is for treating seasonal affective disorder.

In an embodiment of the second aspect (i.e., independently combinable with any of the aspects or embodiments identified herein), the composition is adapted to increase a serum, plasma, or a red blood cell membrane concentration of the C15:0 fatty acid or pharmaceutically acceptable salt thereof to a concentration greater than 2.2 μM and less than 30 μM.

In an embodiment of the second aspect (i.e., independently combinable with any of the aspects or embodiments identified herein), the C15:0 fatty acid or pharmaceutically acceptable salt thereof is pentadecanoic acid.

In an embodiment of the second aspect (i.e., independently combinable with any of the aspects or embodiments identified herein), the unit dosage form comprises from 0.01 mg to 10000 mg of the C15:0 fatty acid or pharmaceutically acceptable salt thereof.

In an embodiment of the second aspect (i.e., independently combinable with any of the aspects or embodiments identified herein), the pharmaceutical composition is substantially free from even chain saturated fatty acids.

In an embodiment of the second aspect (i.e., independently combinable with any of the aspects or embodiments identified herein), the pharmaceutical composition is substantially free from polyunsaturated fatty acids.

In an embodiment of the second aspect (i.e., independently combinable with any of the aspects or embodiments identified herein), the composition is adapted for administration of from 1 mg or less to 500 mg or more, e.g., 0.2 mg to 20 mg, 2.5 mg to 50 mg, e.g., 1.0 to 5.0 mg, e.g., 20 to 500 mg, e.g., 20 to 200 mg, e.g., 100 mg of the C15:0 fatty acid or pharmaceutically acceptable salt thereof, per 1 kg of body weight, per day, to a patient in need thereof. In some embodiments, the effective amount of the C15:0 fatty acid or pharmaceutically acceptable salt thereof in a pharmaceutical composition is from 0.2 to 20 mg/kg body weight.

In an embodiment of the second aspect (i.e., independently combinable with any of the aspects or embodiments identified herein), the unit dosage form is adapted for administration to the patient once per day.

In an embodiment of the second aspect (i.e., independently combinable with any of the aspects or embodiments identified herein), the patient is a human.

In an embodiment of the second aspect (i.e., independently combinable with any of the aspects or embodiments identified herein), the patient is a mammal.

In an embodiment of the second aspect (i.e., independently combinable with any of the aspects or embodiments identified herein), the patient is a domesticated animal.

In an embodiment of the second aspect (i.e., independently combinable with any of the aspects or embodiments identified herein), the domesticated animal is a dog or a cat.

In an embodiment of the second aspect (i.e., independently combinable with any of the aspects or embodiments identified herein), the domesticated animal is a cow, a pig, a sheep, a goat, a horse, a turkey, a duck, or a chicken.

In a generally applicable third aspect (i.e., independently combinable with any of the aspects or embodiments identified herein), a use is provided of a composition for boosting and/or enhancing mood, lowering anxiety and/or pain, treating depression, treating major depressive disorder, or treating seasonal affective disorder, the composition comprising: C15:0 fatty acid or pharmaceutically acceptable salt thereof; and a pharmaceutically acceptable carrier.

In an embodiment of the third aspect (i.e., independently combinable with any of the aspects or embodiments identified herein), the use is for boosting and/or enhancing mood.

In an embodiment of the third aspect (i.e., independently combinable with any of the aspects or embodiments identified herein), the use is for lowering anxiety and/or pain.

In an embodiment of the third aspect (i.e., independently combinable with any of the aspects or embodiments identified herein), the use is for treating depression.

In an embodiment of the third aspect (i.e., independently combinable with any of the aspects or embodiments identified herein), the use is for major depressive disorder.

In an embodiment of the third aspect (i.e., independently combinable with any of the aspects or embodiments identified herein), the use is for s treating seasonal affective disorder.

In an embodiment of the third aspect (i.e., independently combinable with any of the aspects or embodiments identified herein), a serum, plasma, or a red blood cell membrane concentration of the C15:0 fatty acid is increased to a concentration greater than 2.2 μM and less than 30 μM.

In an embodiment of the third aspect (i.e., independently combinable with any of the aspects or embodiments identified herein), the C15:0 fatty acid is pentadecanoic acid.

In an embodiment of the third aspect (i.e., independently combinable with any of the aspects or embodiments identified herein), the C15:0 fatty acid or pharmaceutically acceptable salt thereof is provided as a pharmaceutical composition in a unit dosage form comprising the C15:0 fatty acid or pharmaceutically acceptable salt thereof and a pharmaceutically acceptable carrier.

In an embodiment of the third aspect (i.e., independently combinable with any of the aspects or embodiments identified herein), the unit dosage form comprises from 0.01 mg to 10000 mg of the C15:0 fatty acid or pharmaceutically acceptable salt thereof.

In an embodiment of the third aspect (i.e., independently combinable with any of the aspects or embodiments identified herein), the pharmaceutical composition is substantially free from even chain saturated fatty acids.

In an embodiment of the third aspect (i.e., independently combinable with any of the aspects or embodiments identified herein), the pharmaceutical composition is substantially free from polyunsaturated fatty acids.

In an embodiment of the third aspect (i.e., independently combinable with any of the aspects or embodiments identified herein), the C15:0 fatty acid or pharmaceutically acceptable salt thereof is administered to the patient once per day.

In an embodiment of the third aspect (i.e., independently combinable with any of the aspects or embodiments identified herein), the patient is a human.

In an embodiment of the third aspect (i.e., independently combinable with any of the aspects or embodiments identified herein), the patient is a mammal.

In an embodiment of the third aspect (i.e., independently combinable with any of the aspects or embodiments identified herein), the patient is a domesticated animal.

In an embodiment of the third aspect (i.e., independently combinable with any of the aspects or embodiments identified herein), the domesticated animal is a dog or a cat.

In an embodiment of the third aspect (i.e., independently combinable with any of the aspects or embodiments identified herein), the domesticated animal is a cow, a pig, a sheep, a goat, a horse, a turkey, a duck, or a chicken.

In an embodiment of the third aspect (i.e., independently combinable with any of the aspects or embodiments identified herein), from 1 mg or less to 500 mg or more, e.g., 0.2 mg to 20 mg, 2.5 mg to 50 mg, e.g., 1.0 to 5.0 mg, e.g., 20 to 500 mg, e.g., 20 to 200 mg, e.g., 100 mg, of the C15:0 fatty acid or pharmaceutically acceptable salt thereof, per 1 kg of body weight, per day, is administered to the patient. In some embodiments, the effective amount of the C15:0 fatty acid or pharmaceutically acceptable salt thereof in a pharmaceutical composition is from 0.2 to 20 mg/kg body weight.

In an embodiment of the third aspect (i.e., independently combinable with any of the aspects or embodiments identified herein), the C15:0 fatty acid or pharmaceutically acceptable salt thereof is administered as a component of a food.

Any of the features of an embodiment of the first through sixth aspects is applicable to all aspects and embodiments identified herein. Moreover, any of the features of an embodiment of the first through sixth aspects is independently combinable, partly or wholly with other embodiments described herein in any way, e.g., one, two, or three or more embodiments may be combinable in whole or in part. Further, any of the features of an embodiment of the first through sixth aspects may be made optional to other aspects or embodiments. Any aspect or embodiment of a method or use can be performed using a composition of another aspect or embodiment, and any aspect or embodiment of a composition can be adapted to a method or use of another aspect or embodiment.

### BRIEF DESCRIPTION OF THE DRAWINGS

FIG. 1 illustrates a bar graph of targeted CB1 and CB2 agonist activity of pentadecanoic acid at increasing concentrations.

FIGS. 2A-C illustrate a line graph of the phenotypic profiles of pentadecanoic acid and buproprion HC1.

### DETAILED DESCRIPTION

Compositions and methods for boosting and/or enhancing mood, lowering anxiety and/or pain, treating depression, treating major depressive disorder, or treating seasonal affective disorder are provided. These compositions comprise one or more odd chain saturated fatty acids, derivatives of odd chain saturated fatty acids, or salts thereof, which may be administered in combination with other medicaments or supplements or as part of various treatment regimens as described herein.

### Definitions

The term “alcohol” as used herein is a broad term, and is to be given its ordinary and customary meaning to a person of ordinary skill in the art (and is not to be limited to a special or customized meaning), and refers without limitation to any compound as described herein incorporating one or more hydroxy groups, or being substituted by or functionalized to include one or more hydroxy groups.

The term “derivative” as used herein is a broad term, and is to be given its ordinary and customary meaning to a person of ordinary skill in the art (and is not to be limited to a special or customized meaning), and refers without limitation to any compound as described herein incorporating one or more derivative groups, or being substituted by or functionalized to include one or more derivative groups. Derivatives include but are not limited to esters, amides, anhydrides, acid halides, thioesters, phosphates, triphosphates, and P-sulfenyl derivatives.

The term “hydrocarbon” as used herein is a broad term, and is to be given its ordinary and customary meaning to a person of ordinary skill in the art (and is not to be limited to a special or customized meaning), and refers without limitation to any moiety comprising only carbon and hydrogen atoms. A functionalized or substituted hydrocarbon moiety has one or more substituents as described elsewhere herein.

The term “lipid” as used herein is a broad term, and is to be given its ordinary and customary meaning to a person of ordinary skill in the art (and is not to be limited to a special or customized meaning), and refers without limitation to saturated and unsaturated oils and waxes, derivatives, amides, glycerides, fatty acids, fatty alcohols, sterol and sterol derivatives, phospholipids, ceramides, sphingolipids, tocopherols, and carotenoids, among others.

The terms “pharmaceutically acceptable” as used herein is a broad term, and is to be given its ordinary and customary meaning to a person of ordinary skill in the art (and is not to be limited to a special or customized meaning), and refers without limitation to those compounds, materials, compositions, and/or dosage forms which are, within the scope of sound medical judgment, suitable for contact with the tissues of and/or for consumption by human beings and animals without excessive toxicity, irritation, allergic response, or other problem complications commensurate with a reasonable risk/benefit ratio.

The terms “pharmaceutically acceptable salts” and “a pharmaceutically acceptable salt thereof” as used herein are broad terms, and are to be given their ordinary and customary meaning to a person of ordinary skill in the art (and is not to be limited to a special or customized meaning), and refer without limitation to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′ -dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as, for example, The Merck Index. Any suitable constituent can be selected to make a salt of the therapeutic agents discussed herein, provided that it is non-toxic and does not substantially interfere with the desired activity. In addition to salts, pharmaceutically acceptable precursors and derivatives of the compounds can be employed. Pharmaceutically acceptable amides, lower alkyl derivatives, and protected derivatives can also be suitable for use in compositions and methods of preferred embodiments. While it may be possible to administer the compounds of the preferred embodiments in the form of pharmaceutically acceptable salts, it is generally preferred to administer the compounds in neutral form.

The term “pharmaceutical composition” as used herein is a broad term, and is to be given its ordinary and customary meaning to a person of ordinary skill in the art (and is not to be limited to a special or customized meaning), and refers without limitation to a mixture of one or more compounds disclosed herein with other chemical components, such as diluents or carriers. The pharmaceutical composition facilitates administration of the compound to an organism. Pharmaceutical compositions can also be obtained by reacting compounds with inorganic or organic acids or bases. Pharmaceutical compositions will generally be tailored to the specific intended route of administration.

As used herein, a “carrier” as used herein is a broad term, and is to be given its ordinary and customary meaning to a person of ordinary skill in the art (and is not to be limited to a special or customized meaning), and refers without limitation to a compound that facilitates the incorporation of a compound into cells or tissues. For example, without limitation, dimethyl sulfoxide (DMSO) is a commonly utilized carrier that facilitates the uptake of many organic compounds into cells or tissues of a subject. Water, saline solution, ethanol, and mineral oil are also carriers employed in certain pharmaceutical compositions.

As used herein, a “diluent” as used herein is a broad term, and is to be given its ordinary and customary meaning to a person of ordinary skill in the art (and is not to be limited to a special or customized meaning), and refers without limitation to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” as used herein is a broad term, and is to be given its ordinary and customary meaning to a person of ordinary skill in the art (and is not to be limited to a special or customized meaning), and refers without limitation to a substance that is added to a pharmaceutical composition to provide, without limitation, bulk, consistency, stability, binding ability, lubrication, disintegrating ability etc., to the composition. A “diluent” is a type of excipient.

As used herein, a “subject” as used herein is a broad term, and is to be given its ordinary and customary meaning to a person of ordinary skill in the art (and is not to be limited to a special or customized meaning), and refers without limitation to an animal that is the object of treatment, observation or experiment. “Animal” includes cold- and warm-blooded vertebrates and invertebrates such as fish, shellfish, reptiles and, in particular, mammals. “Mammal” includes, without limitation, dolphins, mice, rats, rabbits, guinea pigs, dogs, cats, sheep, goats, cows, horses, primates, such as monkeys, chimpanzees, and apes, and, in particular, humans. In some embodiments, the subject is human.

As used herein, the terms “treating,” “treatment,” “therapeutic,” or “therapy” are broad terms, and are to be given their ordinary and customary meaning (and are not to be limited to a special or customized meaning) and, without limitation, do not necessarily mean total cure or abolition of the disease or condition. Any alleviation of any undesired markers, signs or symptoms of a disease or condition, to any extent, can be considered treatment and/or therapy. Furthermore, treatment may include acts that may worsen the patient's overall feeling of well-being or appearance.

The terms “therapeutically effective amount” and “effective amount” as used herein are broad terms, and are to be given its ordinary and customary meaning to a person of ordinary skill in the art (and are not to be limited to a special or customized meaning), and are used without limitation to indicate an amount of an active compound, or pharmaceutical agent, that elicits the biological or medicinal response indicated. For example, a therapeutically effective amount of compound can be the amount needed to prevent, alleviate or ameliorate markers or symptoms of a condition or prolong the survival of the subject being treated. This response may occur in a tissue, system, animal or human and includes alleviation of the signs or symptoms of the disease being treated. Determination of a therapeutically effective amount is well within the capability of those skilled in the art, in view of the disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

The term “solvents” as used herein is a broad term, and is to be given its ordinary and customary meaning to a person of ordinary skill in the art (and is not to be limited to a special or customized meaning), and refers without limitation to compounds with some characteristics of solvency for other compounds or means, that can be polar or nonpolar, linear or branched, cyclic or aliphatic, aromatic, naphthenic and that includes but is not limited to: alcohols, derivatives, diesters, ketones, acetates, terpenes, sulfoxides, glycols, paraffins, hydrocarbons, anhydrides, heterocyclics, among others.

As used herein, the phrase “substantially free” means that the composition contains ≤15%, ≤10%, ≤9%, ≤8%, ≤7%, ≤6%, ≤5%, ≤4%, ≤3%, ≤2%, or ≤1%, by weight, of another fatty acid(s).

The term “about,” as used herein, refers to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. When a value is preceded by the term about, the component is not intended to be limited strictly to that value, but it is intended to include amounts that vary from the value.

Any percentages, ratios or other quantities referred to herein are on a weight basis, unless otherwise indicated.

### Odd Chain Fatty Acids

Fatty acids include saturated and unsaturated fatty acids as provided herein, fatty acids are referred to and described using conventional nomenclature as is employed by one of skill in the art. A saturated fatty acid includes no carbon-carbon double bonds. An unsaturated fatty acid includes at least one carbon-carbon double bond. A monounsaturated fatty acid includes only one carbon-carbon double bond. A polyunsaturated fatty acid includes two or more carbon-carbon double bonds. Double bonds in fatty acids are generally cis; however, trans double bonds are also possible. The position of double bonds can be indicated by Δₙ, where n indicates the lower numbered carbon of each pair of double-bonded carbon atoms. A shorthand notation in a form total # carbons # double bonds, Δ_{double bond positions} can be employed. For example, 20:445,8,11,14 refers to a fatty acid having 20 carbon atoms and four double bonds, with the double bonds situated between the 5 and 6 carbon atom, the 8 and 9 carbon atom, the 11 and 12 carbon atom, and the 14 and 15 carbon atom, with carbon atom 1 being the carbon of the carboxylic acid group. Stearate (octadecanoate) is a saturated fatty acid. Oleate (cis-Δ9-octadecenoate) is a monounsaturated fatty acid, linolenate (all-cis-Δ9,12,15-octadecatrienoate) is a polyunsaturated fatty acid. The total number of carbons can be preceded by “C” and double bond positions can be unspecified, e.g., C20:4 referring to a fatty acid having 20 carbon atoms and four double bonds.

A fatty acid may be referred to by various names, for example, heptadecanoic acid may be referred to as heptadecylic acid, margaric acid, and n-heptadecylic acid, or C17:0. A fatty acid may be referred to by lipid numbers, as known in the art.

In some embodiments, the fatty acid can be an odd chain saturated fatty acid. In further embodiments, one or more fatty acids can include at least one odd chain saturated fatty acid.

Examples of odd chain fatty acids are margaric acid (heptadecanoic acid, C17:0), pelargonate (nonanoic acid, C9:0), undecanoic acid (C11:0), nonadecanoic acid (C19:0), pentadecanoic acid (C15:0), arachidonate ((5Z,8Z,11Z,14Z)-icosa-5,8,11,14-tetraenoic acid), adrenate (all-cis-7,10,13,16-docosatetraenoic acid), and osbond acid (all-cis-4,7,10,13,16-docosapentaenoic acid). Generally, the one or more odd chain fatty acids have from 9 carbon atoms to 31 carbon atoms (9, 11, 13, 15, 17, 19, 21, 23, 25, 27, 29, or 31 carbon atoms), for example, from 15 to 21 carbon atoms, for example 17 carbon atoms; however, in certain embodiments higher or lower odd numbers of carbon atoms can be acceptable. Generally, the one or more odd chain fatty acids are saturated; however, in certain embodiments mono or polyunsaturated odd chain fatty acids can be acceptable.

An odd chain fatty acid may include saturated or unsaturated hydrocarbon chains. An odd chain fatty acid may be present as a carboxylic derivative. An odd chain fatty acid may be present as a salt, for example, at the carboxylic group. In some embodiments, one odd chain fatty acid may be present, two odd chain fatty acids may be present, three odd chain fatty acids may be present, or more. In some embodiments, odd chain fatty acids in a mixture including a plurality of odd chain fatty acids may be distinguished by the amount of unsaturation, the length of the hydrocarbon chain, varying states of derivativeification, or by other structural features.

Odd chain fatty acids are found in trace amounts in some dairy products, including butter (see, e.g., Mansson HL (2008), Fatty acids in bovine milk fat, Food Nutr. Res. 52:4). Studies have demonstrated that increasing daily dietary intake of foods with odd chain fatty acids successfully increases serum or plasma levels (see, e.g., Benatar J.R., Stewart R.A.H. (2014), The effects of changing dairy intake on trans and saturated fatty acid levels—results from a randomized controlled study. Nutr. J. 13:32).

Generally, a fatty acid, such as an odd chain fatty acid can be provided as a free fatty acid, or a derivative thereof. Such derivatives include, but are not limited to, acyl glycerides. An acyl glyceride may be substituted with up to three acyl fatty acid esters. Thus, an acyl glyceride can be a monoacylglyceride (MAG), diacylglyceride (DAG), or a triacylglyceride (TAG). The glyceride can include more than one type of fatty acid ester. For example, a glyceride can include a heptdecanoate and a docosanoate. A glyceride can also be a structured triacylglyceride (STAG), a plasmalogen, or a phospholipid. The fatty acid ester can be in the snl position or the sn2 position, or both positions. The snl and sn2 positions can be substituted by the same or different fatty acid esters. As a non-limiting example, a structured triacylglyceride can be sn-1,3-C17-sn-2-oleoyl.

In some embodiments, a fatty acid can be provided as a free fatty acid, a cholesterol ester, a glycerol ester (including, but not limited to a monoacylglyceride (MAG), diacylglyceride (DAG), or a triacylglyceride (TAG)), a phospholipid (including, but not limited to, a phosphatidylcholine, a lysophosphatidylcholine, a phosphatidylethanolamine, a lysophosphatidylethanolamine, or a phosphatidylserine), a ceramide (including but not limited to a hexosyl ceramide) or a sphingolipid. A non-limiting example of a phophatidylcholine is 2,3-di-C17:0-phosphatidylcholine. A non-limiting example of a lysophophatidylcholine is 2-lyso-3-C17:0-phosphatidylcholine. In some embodiments, a derivative of a fatty acid can be a β-sulfenyl derivative. It is thought that β-sulfenyl derivative, such as an acid or ester, can be resistant to δ-oxidation in the body. As a non-limiting example, the δ-sulfenyl derivative of heptadecanoic acid is tetradecylthioacetic acid. Derivatives can be synthesized by standard methods known to those of skill in the art.

In some embodiments, a fatty acid may be provided as a constituent of a specific type of lipid, for example, a ceramide, a phospholipid, a sphingolipid, a membrane lipid, a glycolipid, or a triglyceride.

In some embodiments, a fatty acid, such as a very long even chain fatty acid, is provided in a bioavailable form. The term “bioavailability” refers to the fraction of an administered dose of unchanged drug that reaches the systemic circulation, one of the principal pharmacokinetic properties of drugs. By definition, when a medication is administered intravenously, its bioavailability is 100%. As employed herein, the term “bioavailable” refers to a form of the fatty acid that is successfully absorbed by the body when using methods of administration other than intravenous, for example, an oral therapeutic). In some embodiments, very long even chain fatty acid-based compositions may include adaptions that optimize absorption. In some embodiments, a very long even chain fatty acid can be provided as a structured triacylglyceride. In further embodiments, the fatty acid is in the sn-2 position of a structured triacylglyceride.

A pure or purified fatty acid may exist in various physical states. For example, heptadecanoic acid exists as an off-white powder that is stable at room temperature; this compound can be purchased in forms suitable for research purposes in small amounts from some commercial suppliers (for example, from Sigma-Aldrich corp., of St. Louis, MO). Other fatty acids, or salts or derivatives thereof, may exist as oils, solids, crystalline solids, or gases.

An odd chain fatty acid or the pharmaceutically acceptable salts or derivatives thereof, may be provided in a purity (e.g., a percentage of the fatty acid, or its pharmaceutically acceptable salts or derivatives, in a bulk form) of at least about 10%, at least about 20%, at least about 30%, at least about 40%, at least about 50%, at least about 60%, at least about 70%, at least about 80%, at least about 90%, at least about 95%, at least about 98%, at least about 99%, at least about 99.9%, at least about 99.99%, or substantially pure, wherein substantially pure may include, but not be limited to, a product with impurities at a level such that no physiological effect from the presence of the impurities is detectable. A mixture of fatty acids, such as, for example, odd chain fatty acids and/or very long even chain fatty acids, or pharmaceutically acceptable salts or derivatives thereof, may be present in a purity of at least about 10%, at least about 20%, at least about 30%, at least about 40%, at least about 50%, at least about 60%, at least about 70%, at least about 80%, at least about 90%, at least about 95%, at least about 98%, at least about 99%, at least about 99.9%, at least about 99.99%, or substantially pure. The fatty acid, or a mixture thereof, or a pharmaceutically acceptable salt or derivative thereof, may be free from other fatty acids or fatty acid derivatives, may be free from triglycerides, or may be free from phospholipids. Without limitation, an odd chain fatty acid as provided herein may be substantially free from even chain fatty acids, singly or taken as a group; even chain fatty acids include, for example, myristic acid (C14:0), palmitic acid (C16:0), or stearic acid (C18:0). In some embodiments, an odd chain fatty acid as provided herein may be substantially free from short-chain fatty acids (SCFA, e.g., a fatty acid with 2-6 carbon atoms), medium-chain fatty acids (MCFA, e.g., a fatty acid with 7-12 carbon atoms), long-chain fatty acids (LCFA, e.g., a fatty acid with 13-22 carbon atoms), or very long chain fatty acids (VLCFA, e.g., a fatty acid with 23 or more carbon atoms).

A fatty acid, such as an odd chain fatty acid or a pharmaceutically acceptable salt or derivative thereof, may be from any source. In some embodiments, a fatty acid, or its pharmaceutically acceptable salts or derivatives, may be present in natural sources, may be isolated from natural sources, may be semi-synthetic, may be synthetic, or may be a mixture of one or more of these. The fatty acid, or its pharmaceutically acceptable salts or derivatives, may be produced in a laboratory, may be produced in nature, may be produced by enzymatic processes, may be produced by wild microbes, may be produced by genetically modified microbes, may be isolated from animal tissues, may be produced by chemical synthesis, or may be produced by a plurality of these processes.

The fatty acid may be derived from natural sources, e.g., fish oils, or can be synthesized by methods as are known in the art. In some embodiments, the fatty acid may be contaminated with undesired components present in unrefined or unpurified natural products. In such situations, it can be desirable to remove undesired components, or to increase the concentration of desired components using known separation or purification techniques.

In any compound described, all tautomeric forms are also intended to be included. Without limitation, all tautomers of carboxylic groups are intended to be included.

In any compound described herein having one or more double bond(s) generating geometrical isomers that can be defined as E or Z, each double bond may independently be E or Z, or a mixture thereof.

Where compounds disclosed herein have unfilled valencies, then the valencies are to be filled with hydrogens or isotopes thereof, e.g., hydrogen-1 (protium) and hydrogen-2 (deuterium).

The fatty acid, such as an odd chain fatty acid, as described herein, includes crystalline forms (also known as polymorphs, which include the different crystal packing arrangements of the same elemental composition of a compound), amorphous phases, salts, solvates, and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

The compounds described herein can be labeled isotopically. In some circumstances, substitution with isotopes such as deuterium may afford certain therapeutic advantages resulting from greater metabolic stability, such as, for example, increased in vivo half-life or reduced dosage requirements. Isotopic substitution may be beneficial in monitoring subject response to administration of a compound, for example, by providing opportunity for monitoring of the fate of an atom in a compound. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

The prevalence of various fatty acids in the diet has been correlated to the occurrence of metabolic syndrome in subjects (see, e.g., Forouhi N, Koulman A, Sharp S, Imamura F, Kroger J, Schulze M, et al. (2014), Differences in the prospective association between individual plasma phospholipid saturated fatty acids and incident type 2 diabetes: the EPIC-InterAct case-cohort study. Lancet Diabetes Endocrinol. 2:810-8). Indeed, whole-fat dairy consumption has been correlated with a decreased risk of metabolic syndrome markers (see, e.g., Kratz M, Marcovina S, Nelson JE, Yeh MM, Kowdley KV, Callahan HS, et al. (2014), Dairy fat intake is associated with glucose tolerance, hepatic and systemic insulin sensitivity, and liver fat but not beta-cell function in humans, Am. J. Clin. Nutr., 99:1385-96).

The mechanism(s) by which odd chain saturated fatty acid(s) have a beneficial effect are not well understood. Without wishing to be limited by theory, it is thought that fatty acids, or derivatives thereof, can be elongated (increased in chain length) or chain shortened by metabolic processes in the body, to form different fatty acids, or derivatives thereof. Peroxidation of certain fatty acids may create products with signaling characteristics in the body. It is thought that fatty acids of certain chain length create signaling products that substantially contribute to one or more conditions provided herein. In some embodiments, an odd chain fatty acid is elongated to form a very long chain fatty acid, such as a very long even chain fatty acid. In further embodiments, a very long even chain fatty acid can be chain-shortened to an odd chain fatty acid. Levels of very long even chain fatty acids in the body may increase following administration of one or more odd chain fatty acids. Levels of odd chain fatty acids in the body may increase following administration of one or more very long even chain fatty acids. Pharmaceutical Compositions Including One or More Fatty Acids

Formulations including a fatty acid, such as an odd chain fatty acid or a very long even chain fatty acid, or a salt or derivative thereof, and at least one excipient are provided. It is generally preferred to administer the compounds of the embodiments in oral formulations; however, other routes of administration are also contemplated.

The pharmaceutical compositions described herein can be administered by themselves to a subject, or in compositions where they are mixed with other active agents, as in combination therapy, or with carriers, diluents, excipients or combinations thereof. Formulation is dependent upon the route of administration chosen. Techniques for formulation and administration of the compounds described herein are known to those skilled in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The pharmaceutical compositions disclosed herein may be manufactured by a process that is itself known, e.g., by means of conventional mixing, dissolving, granulating, dragee-making, levigating, emulsifying, encapsulating, entrapping, tableting, or extracting processes. Many of the compounds used in the pharmaceutical combinations disclosed herein may be provided as salts with pharmaceutically acceptable counterions.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections. Contemplated herein is any combination of the forgoing, or other methods as would be known to one of ordinary skill in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

In practice, a fatty acid, such as an odd chain saturated fatty acid or a salt or derivative thereof, may be combined as the active ingredient in intimate admixture with a pharmaceutical carrier according to conventional pharmaceutical compounding techniques. The carrier can take a wide variety of forms depending on the form of preparation desired for administration. Thus, the pharmaceutical compositions provided herein can be presented as discrete units suitable for oral administration such as capsules, cachets or tablets each containing a predetermined amount of the active ingredient. Further, the compositions can be presented as an oil, a powder, as granules, as a solution, as a suspension in an aqueous liquid, as a non-aqueous liquid, as an oil-in-water emulsion, or as a water-in-oil liquid emulsion. In addition to the common dosage forms set out above, the compounds provided herein, or pharmaceutically acceptable salts or derivatives thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

A formulation may also be administered in a local rather than systemic manner, for example, via injection of the compound directly into the infected area, often in a depot or sustained release formulation. Furthermore, a targeted drug delivery system might be used, for example, in a liposome coated with a tissue specific antibody.

The pharmaceutical compositions may contain a fatty acid, such as an odd chain fatty acid, or a salt or derivative thereof, in an amount effective for the desired therapeutic effect. In some embodiments, the pharmaceutical compositions are in a unit dosage form and comprise from about 1 mg or less to about 5000 mg or more per unit dosage form. In further embodiments, the pharmaceutical compositions comprise from about 1 to about 500 mg per unit dosage form or from about 500 to 5000 mg per unit dosage form. Such dosage forms may be solid, semisolid, liquid, an emulsion, or adapted for delivery via aerosol or the like for inhalation administration.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, lower alcohols, and water. Examples of gaseous carriers include carbon dioxide and nitrogen.

Pharmaceutical compositions provided herein can be prepared as solutions or suspensions of the active compound(s) in water. A suitable surfactant can be included such as, for example, hydroxypropylcellulose. Dispersions can also be prepared in glycerol, liquid polyethylene glycols, and mixtures thereof in oils. Further, a preservative can be included to, for example, prevent the detrimental growth of microorganisms.

Pharmaceutical compositions provided herein suitable for injectable use include sterile aqueous solutions or dispersions. Furthermore, the compositions can be in the form of sterile powders for the extemporaneous preparation of such sterile injectable solutions or dispersions. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound provided herein, or pharmaceutically acceptable salt or derivative thereof, can also be prepared in powder or liquid concentrate form for dilution.

The fatty acid, such as an odd chain saturated fatty acid, or a salt or derivative thereof, can be formulated as a liposome. The fatty acid can be a component of the lipid portion of the liposome or can be encapsulated in the aqueous portion of the liposome. The fatty acid, such as an odd chain fatty acid, or a salt or derivative thereof, can also be coformulated with a cyclodextrin. The cyclodextrin can be, for example, hydroxypropyl-β-cyclodextrin or a sulfobutylether cyclodextrin.

Contemplated herein are compositions including a fatty acid, such as an odd chain saturated fatty acid, or a salt or derivative thereof in combination with at least one additional active agent. A fatty acid, such as an odd chain saturated fatty acid, or a salt or derivative thereof, and the at least one additional active agent(s) may be present in a single formulation or in multiple formulations provided together, or may be unformulated (for example, free of excipients and carriers). In some embodiments, a fatty acid, such as an odd chain saturated fatty acid, or a salt or derivative thereof, can be administered with one or more additional agents together in a single composition. For example, a compound of a fatty acid, such as an odd chain saturated fatty acid, or a salt or derivative thereof, can be administered in one composition, and at least one of the additional agents can be administered in a second composition. In a further embodiment, a fatty acid, such as an odd chain saturated fatty acid, or a salt or derivative thereof and the at least one additional active agent(s) are co-packaged in a kit. For example, a drug manufacturer, a drug reseller, a physician, a compounding shop, or a pharmacist can provide a kit comprising a disclosed compound or product and another component for delivery to a patient.

Some embodiments described herein relate to a pharmaceutical composition, which can include a therapeutically effective amount of one or more compounds described herein (e.g., a fatty acid, such as an odd chain saturated fatty acid or a pharmaceutically acceptable salt or derivative thereof) and a pharmaceutically acceptable carrier, diluent, excipient or combination thereof. The pharmaceutical composition can include a fatty acid such as an odd chain saturated fatty acid, or a salt or derivative thereof in, for example, >1%, ≥2%, ≥3%, ≥4%, ≥5%, ≥6%, ≥7%, ≥8%, ≥9%, ≥10%, ≥20%, ≥30%, ≥40%, ≥50%, ≥60%, ≥70%, ≥80%, ≥90%, ≥95%, or ≥98% of the composition. In some embodiments, the pharmaceutical composition can include a plurality of fatty acids, such as one or more of an odd chain saturated fatty acid and/or a very long even chain fatty acid, or salts or derivatives thereof in, for example, ≥1%, ≥2%, ≥3%, ≥4%, ≥5%, ≥6%, ≥7%, ≥8%, ≥9%, ≥10%, ≥20%, ≥30%, ≥40%, ≥50%, ≥60%, ≥70%, ≥80%, ≥90%, ≥95%, or ≥98% of the composition. Foodstuffs

Foodstuffs, dietary supplements, and other comestibles including a fatty acid, such as an odd chain saturated fatty acid, or a salt or derivative thereof, are provided, wherein an amount of the fatty acid in the foodstuff has been fortified (e.g., enriched or concentrated). A fatty acid, such as an odd chain saturated fatty acid, provided herein may be added to foodstuffs for consumption by a subject. The fatty acid, such as an odd chain saturated fatty acid, may be integrated into one or more ingredients of a foodstuff. The fatty acid, such as an odd chain saturated fatty acid, may be prepared as an ingredient, or may be unprepared. The compound, or preparation including the compound, may be added prior to preparation, during preparation, or following preparation. Preparation may without limitation include cooking, mixing, flavoring, seasoning, blending, boiling, frying, baking, or other processes known in the art. Fortification is preferably at a level so as to provide a therapeutic daily dosage of the fatty acid as described elsewhere herein; however, beneficial effects may also be obtained at amounts below such dosages.

A fatty acid, such as an odd chain saturated fatty acid, or salt or derivative thereof, as provided herein may be present as a constituency in foodstuffs by operation of processes known in nature, for example, by altering the metabolic processes of a plant, animal, bacteria, or fungus. Genetic alteration of a plant, animal, bacteria, or fungus to increase the concentration of a fatty acid, such as an odd chain saturated fatty acid, or a salt or derivative thereof, is contemplated. By way of example, the fatty acid can be present in the foodstuff in a concentration of at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, or higher, for example, 1% to 2% or 3% or 4% or 5% or 6% or 7% or 8% or 9% or 10% or 20% or 30% or 40% or 50%.

### Indications

Provided are compositions and methods for supporting a healthy body weight, supporting maintenance of body mass index, promoting a reduction in body mass index, promoting satiety, promoting reduced calorie consumption, and/or promoting efficient fat metabolism are provided. These compositions comprise one or more odd chain saturated fatty acids, derivatives of odd chain saturated fatty acids, or salts thereof, which may be administered in combination with other medicaments or supplements or as part of various treatment regimens as described herein. Pentadecanoic acid has these activities in humans at daily oral doses ranging from 1 mg/day or less to 500 mg/day or more per day, e.g., 0.2 mg/day to 20 mg/day, 1.0 mg/day to 5.0 mg/day, 5 mg/day to 50 mg/day, 20 mg/day to 200 mg/day, e.g., 100 mg/day etc., as described herein.

Without wishing to be limited by theory, it is thought that increasing odd chain saturated fatty acid free fatty acid or phospholipid levels in the serum, plasma, and cells to targeted concentrations may boost and/or enhance mood, lower anxiety and/or pain, treat depression, treat major depressive disorder, or treat seasonal affective disorder.

In some embodiments, the methods provided herein increase levels of serum, plasma, or erythrocyte membrane odd chain fatty acids.

In some embodiments, levels of serum, plasma, or erythrocyte membrane very long even chain fatty acids may increase following administration of one or more odd chain fatty acids, or a salt or derivative thereof.

Provided herein are methods for treating including the step of administering a dose of a fatty acid, such as an odd chain fatty acid or a very long even chain fatty acid, at a predetermined interval, or at an interval left to the discretion of the subject.

In some embodiments, the compounds and methods provided herein may provide a threshold serum, plasma, or red blood cell membrane percentage of an odd chain fatty acid relative to all serum, plasma, or red blood cell membrane fatty acids, respectively. For example, the threshold value may be a value of about 0.05% or lower to 90% or higher, e.g., a value of at least about 0.05%, at least about 0.1%, at least about 0.2%, at least about 0.3%, at least about 0.4%, at least about 0.5%, at least about 0.6%, at least about 0.7%, at least about 0.8%, at least about 0.9%, at least about 1.0%, at least about 1.1%, at least about 1.2%, at least about 1.3%, at least about 1.4%, at least about 1.5%, at least about 1.6%, at least about 1.7%, at least about 1.8%, at least about 1.9%, at least about 2.1%, at least about 2.2%, at least about 2.3%, at least about 2.4%, at least about 2.5%, at least about 2.6%, at least about 2.7%, at least about 2.8%, at least about 2.9%, at least about 3.0%, at least about 3.5%, at least about 4.0%, at least about 4.5%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or more than 90%.

In some embodiments, the compounds and methods provided herein may provide an increase above a baseline value (e.g., pretreatment value in a patient being treated, or general value observed in a particular patient population) in a serum or plasma concentration of an odd chain fatty acid, or red blood cell membrane concentration of an odd chain fatty acid. For example, a serum or plasma odd chain fatty acid or red blood cell membrane concentration of an odd chain fatty acid may be increased by at least about 1 μg/ml, at least about 2 μg/ml, at least about 3 μg/ml, at least about 4 μg/ml, at least about 5 μg/ml, at least about 6 μg/ml, at least about 7 μg/ml, at least about 8 μg/ml, at least about 9 μg/ml, at least about 10 μg/ml, at least about 15 μg/ml, at least about 20 μg/ml, at least about 25 μg/ml, at least about 30 μg/ml, at least about 35 μg/ml, at least about 40 μg/ml, at least about 45 μg/ml, at least about 50 μg/ml, or more than 50 μg/ml. In some embodiments, the serum concentration of an odd chain fatty acid, or red blood cell membrane concentration of an odd chain fatty acid may increase above a baseline value (e.g., pretreatment value in a patient being treated, or general value observed in a particular patient population) by at least about 0.01×10⁻⁴ M, at least about 0.05×10⁻⁴ M, at least about 0.1×10⁻⁴ M, at least about 0.2×10⁻⁴ M, at least about 0.3×10⁻⁴ M, at least about 0.4×10⁻⁴ M, at least about 0.5×10⁻⁴ M, at least about 0.6×10⁻⁴ M, at least about 0.7×10⁻⁴ M, at least about 0.8×10⁻⁴ M, at least about 0.9×10⁻⁴ M, at least about 1×10⁻⁴ M, at least about 2×10⁻⁴ M, or at least about 3×10⁻⁴ M.

In some embodiments, the compounds and methods provided herein may provide an increase in serum or plasma total odd chain fatty acids, or red blood cell membrane total odd chain fatty acids. For example, serum total odd chain fatty acids, or red blood cell membrane total odd chain fatty acids, may be increased above a baseline value (e.g., pretreatment value in a patient being treated, or general value observed in a particular patient population) by at least about 5 μg/ml, at least about 6 μg/ml, at least about 7 μg/ml, at least about 8 μg/ml, at least about 9 μg/ml, at least about 10 μg/ml, at least about 15 μg/ml, at least about 20 μg/ml, at least about 25 μg/ml, at least about 30 μg/ml, at least about 35 μg/ml, at least about 40 μg/ml, at least about 45 μg/ml, at least about 50 μg/ml, at least about 60 μg/ml, at least about 70 μg/ml, at least about 80 μg/ml, at least about 90 μg/ml, at least about 100 μg/ml, at least about 150 μg/ml, at least about 200 μg/ml, at least about 250 μg/ml, at least about 300 μg/ml, at least about 350 μg/ml, at least about 400 μg/ml, at least about 450 μg/ml, at least about 500 μg/ml, or more than 500 μg/ml.

In some embodiments, the compounds and methods provided herein may provide an increase above a baseline value (e.g., pretreatment value in a patient being treated, or general value observed in a particular patient population) in a serum, plasma, or red blood cell membrane odd chain fatty acids relative to all serum or red blood cell membrane fatty acids, respectively. For example, a serum, plasma, or red blood cell membrane odd chain fatty acid may be increased above a baseline value (e.g., pretreatment value in a patient being treated, or general value observed in a particular patient population) by at least about 0.01%, at least about at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 1.1%, at least about 1.2%, at least about 1.3%, at least about 1.4%, at least about 1.5%, at least about 1.6%, at least about 1.7%, at least about 1.8%, at least about 1.9%, at least about 2%, at least about 2.1%, at least about 2.2%, at least about 2.3%, at least about 2.4%, at least about 2.5%, at least about 2.6%, at least about 2.7%, at least about 2.8%, at least about 2.9%, at least about 3%, at least about 3.5%, at least about 4%, at least about 4.5%, at least about 5%, or more than 5%.

In some embodiments, the compounds and methods provided herein may provide a reduction in elevated erythrocyte sedimentation rate.

In some embodiments, the compounds and methods provided herein may provide a reduction in elevated alkaline phosphatase.

In some embodiments, the compounds and methods provided herein may provide a reduction in serum ferritin. For example, serum ferritin may be reduced below a baseline value (e.g., pretreatment value in a patient being treated, or general value observed in a particular patient population) by at least about 10 ng/ml, at least about 100 ng/ml, at least about 200 ng/ml, at least about 300 ng/ml, at least about 400 ng/ml, at least about 500 ng/ml, at least about 600 ng/ml, at least about 700 ng/ml, at least about 800 ng/ml, at least about 900 ng/ml, at least about 1000 ng/ml, at least about 1100 ng/ml, at least about 1200 ng/ml, at least about 1300 ng/ml, at least about 1400 ng/ml, at least about 1500 ng/ml, at least about 2000 ng/ml, at least about 2500 ng/ml, at least about 3000 ng/ml, at least about 3500 ng/ml, at least about 4000 ng/ml, at least about 4500 ng/ml, at least about 5000 ng/ml, at least about 6000 ng/ml, at least about 7000 ng/ml, at least about 8000 ng/ml, at least about 9000 ng/ml, at least about 10000 ng/ml, or more than 10000 ng/ml.

In some embodiments, the compounds and methods provided herein may provide a reduction in serum ferritin below a specified level. For example, serum ferritin may be reduced below about 20000 ng/ml, about 15000 ng/ml, about 12000 ng/ml, about 10000 ng/ml, about 8000 ng/ml, about 5000 ng/ml, about 2000 ng/ml, about 1000 ng/ml, or about 500 ng

In some embodiments, an odd chain fatty acid (e.g., a saturated odd chain fatty acid) is administered to maintain serum or plasma total percent of the odd chain fatty acid, or all odd chain fatty acids, above a predetermined threshold value. In variations of these embodiments, the odd chain fatty acid is heptadecanoic acid. In further variations, the odd chain fatty acid is administered to maintain serum phospholipid percent of the odd chain fatty acid, or all odd chain fatty acids, above about 0.1%, about 0.2%, about 0.3%, about 0.4%, about 0.5%, about 0.6%, about 0.7%, about 0.8%, about 0.9%, about 1%, about 1.2%, about 1.4%, about 1.6%, about 1.8%, about 2%, about 2.2%, about 2.4%, or about 2.6%.

In some embodiments, the compounds and methods provided herein may provide a threshold serum, plasma, or red blood cell membrane percentage of a very long even chain fatty acid relative to all serum or red blood cell membrane fatty acids, respectively. For example, the threshold value may be a value of about 0.05% or lower to 90% or higher, e.g., a value of at least about 0.05%, at least about 0.1%, at least about 0.2%, at least about 0.3%, at least about 0.4%, at least about 0.5%, at least about 0.6%, at least about 0.7%, at least about at least about 0.9%, at least about 1.0%, at least about 1.1%, at least about 1.2%, at least about 1.3%, at least about 1.4%, at least about 1.5%, at least about 1.6%, at least about 1.7%, at least about 1.8%, at least about 1.9%, at least about 2.1%, at least about 2.2%, at least about 2.3%, at least about 2.4%, at least about 2.5%, at least about 2.6%, at least about 2.7%, at least about 2.8%, at least about 2.9%, at least about 3.0%, at least about 3.5%, at least about 4.0%, at least about 4.5%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or more than 90%.

In some embodiments, the compounds and methods provided herein may provide an increase above a baseline value (e.g., pretreatment value in a patient being treated, or general value observed in a particular patient population) in a serum or plasma concentration of a very long even chain fatty acid, or red blood cell membrane concentration of a very long even chain fatty acid. For example, a serum very long even chain fatty acid or red blood cell membrane concentration of a very long even chain fatty acid may be increased by at least about 0.01 μg/ml, at least about 0.05 μg/ml, at least about 0.1 μg/ml, at least about 0.4 μg/ml, 1 μg/ml, at least about 2 μg/ml, at least about 3 μg/ml, at least about 4 μg/ml, at least about 5 μg/ml, at least about 6 μg/ml, at least about 7 μg/ml, at least about 8 μg/ml, at least about 9 μg/ml, at least about 10 μg/ml, at least about 15 μg/ml, at least about 20 μg/ml, at least about 25 μg/ml, at least about 30 μg/ml, at least about 35 μg/ml, at least about 40 μg/ml, at least about 45 μg/ml, at least about 50 μg/ml, or more than 50 μg/ml. In some embodiments, the serum concentration of a very long even chain fatty acid, or red blood cell membrane concentration of a very long even chain fatty acid may increase above a baseline value (e.g., pretreatment value in a patient being treated, or general value observed in a particular patient population) by at least about 0.001×10⁻⁴ M, at least about 0.005×10⁻⁴ M, at least about 0.05×10⁻⁴ M, at least about 0.01×10⁻⁴ M, at least about 4 M, at least about 0.1x10 4 M, at least about 0.2×10⁻⁴ M, at least about 0.3×10⁻⁴ M, at least about 0.4×10⁻⁴ M, at least about 0.5×10⁻⁴ M, at least about 0.6×10⁻⁴ M, at least about 0.7×10⁻⁴ M, at least about 0.8×10⁻⁴ M, at least about 0.9×10⁻⁴ M, at least about 1×10⁻⁴ M, at least about 2×10⁻⁴ M, or at least about 3×10⁻⁴ M.

In some embodiments, the compounds and methods provided herein may provide an increase in serum or plasma total very long even chain fatty acids, or red blood cell membrane total very long even chain fatty acids. For example, serum total very long even chain fatty acids, or red blood cell membrane total very long even chain fatty acids, may be increased above a baseline value (e.g., pretreatment value in a patient being treated, or general value observed in a particular patient population) by at least about 0.05 μg/ml, at least about 0.1 μg/ml, at least about 0.5 μg/ml, at least about 1 μg/ml, at least about 5 μg/ml, at least about 6 μg/ml, at least about 7 μg/ml, at least about 8 μg/ml, at least about 9 μg/ml, at least about 10 μg/ml, at least about 15 μg/ml, at least about 20 μg/ml, at least about 25 μg/ml, at least about 30 μg/ml, at least about 35 μg/ml, at least about 40 μg/ml, at least about 45 μg/ml, at least about 50 μg/ml, at least about 60 μg/ml, at least about 70 μg/ml, at least about 80 μg/ml, at least about 90 μg/ml, at least about 100 μg/ml, at least about 150 μg/ml, at least about 200 μg/ml, at least about 250 μg/ml, at least about 300 μg/ml, at least about 350 μg/ml, at least about 400 μg/ml, at least about 450 μg/ml, at least about 500 μg/ml, or more than 500 μg/ml.

In some embodiments, a composition or method provided herein may provide an increase in red blood cell count. For example, a red blood cell count level may be increased above a baseline value (e.g., pretreatment value in a patient being treated, or general value observed in a particular patient population) by at least about 0.1 cells/μL, at least about 0.2 cells/μL, at least about 0.3 cells/μL, at least about 0.4 cells/μL, at least about 0.5 cells/μL, at least about 0.6 cells/μL, at least about 0.7 cells/μL, at least about 0.8 cells/μL, at least about 0.9 cells/μL, at least about 1 cell/μL, at least about 1.2 cells/μL, at least about 1.4 cells/μL, at least about 1.6 cells/μL, or at least about 2 cells/III— Combination Therapies

In some embodiments, the compounds disclosed herein, such as an odd chain fatty acid, or a salt or derivative thereof, or a very long even chain fatty acid, or a salt or derivative thereof, or a pharmaceutical composition that includes a compound described herein, or a salt or derivative thereof, may be used in combination with one or more additional active agents. Examples of additional active agents that can be used in combination with a compound of an odd chain fatty acid, or a salt or derivative thereof, or a composition that includes a compound of an odd chain fatty acid, or a salt or derivative thereof, include, but are not limited to, agents currently used for treating conditions provided herein, and as otherwise known to medical science.

In some embodiments, a compound of an odd chain fatty acid, or a salt or derivative thereof, or a composition that includes a compound of an odd chain fatty acid, or a salt or derivative thereof, can be used with one, two, three or more additional active agents described herein. Such agents include, but are not limited to, a second fatty acid, such as an odd chain fatty acid or a very long even chain fatty acid, or a salt or derivative thereof. In some embodiments, a composition can include at least one odd chain fatty acid, or a salt or derivative thereof, and at least one very long even chain fatty acid, or a salt or derivative thereof.

In some embodiments, a compound of an odd chain fatty acid, or a salt or derivative thereof, or a composition that includes a compound of an odd chain fatty acid, or a salt or derivative thereof, can be used (for example, administered or ingested) in combination with another agent or agents for boosting and/or enhancing mood, lowering anxiety and/or pain, treating depression, treating major depressive disorder, or treating seasonal affective disorder.

For example, a compound of a fatty acid, such as an odd chain fatty acid, disclosed herein can be used in combination with one or more agents selected from stimulants (e.g., caffeine), statins (e.g., atorvastatin (Lipitor), fluvastatin (Lescol), lovastatin (Altoprev), pitavastatin (Livalo), pravastatin (Pravachol), rosuvastatin (Crestor), simvastatin (Zocor)), cholesterol absorbers (e.g., ezetimibe (Zetia)), bile acid sequestrants (e.g., cholestyramine (Prevalite), colesevelam (Welchol), colestipol (Colestid)), ezetimibe-simvastatin (Vytorin), alirocumab (Praluent), evolocumab (Repatha), PKSK9 inhibitors, fibrates (e.g., fenofibrate (Tricor), gemfibrozil (Lopid)), niacin, phytosterols, and fish oils/omega 3 fatty acids, weight loss medications (orlistat (Xenical), lorcaserin (Belviq), phentermine and topiramate (Qsymia), buproprion and naltrexone (Contrave), liraglutide (Saxenda), phentermine, Adipex-P, Topamax, Desoxyn, Alli, Xenical, phendimetrazine, Tenuate, Fastin, Qsymia, bupropion, diethylpropion, HCG, methamphetamine, Bontril Slow Release, Didrex, lorcaserin, Saxenda, lonamin, Pregnyl, Bontril PDM, chorionic gonadotropin, phentermine/topiramate, Tagamet, Topiragen, Zantryl, liraglutide, T-Diet, Topamax Sprinkle, amphetamine, benzphetamine, bupropion/naltrexone, cimetidine, Evekeo, methylphenidate, Suprenza, Tenuate Dospan, Adipost, Atti-Plex P, Belviq XR, desvenlafaxine, Lomaira, Oby-Cap, Phendiet, Phentercot, Phentride, Prelu-2, Tagamet HB, Equaline Acid Reducter, Melfiat, Obezine, Phendiet-105, Recede, Regimex, Tepanil, Garcinia, Guarana, Hoodia, Ephedra), anticoagulants (Heparin, Warfarin, Apixaban, Dabigatran, Dalteparin, Edoxaban, Enoxaparin, Fondaparinux, Rivaroxaban, Betrixaban), Xa inhibitors, anti-fibrotics (nintedanib, pirfenidone, epinephrine, NSAIDs, antihistamines, corticosteroids (cortisone, predinsone), cyclophosphamide, azathioprine, micophenolate mofetil, N-acetylcysteine, proton pump inhibitors (Prilosec, Nexium), bronchodilators (albuterol, theophilline, ipratropium), mucolytics (guaifenesin, Dnase, N-acetylcysteine, hypertonic saline), anti-inflammatives (triamcinolone, flunisolide, fluticasone, beclomethasone, prednisone, methylprednisone, ibuprofen, montelukast, cromolyn, N-acetylcysteine), antibiotics (ciprofloxacin, co-trimoxazole, tobramycin, cephalexin, colistin, dicloxaccillin, azithromycin, amoxicillin, cipro, levofloxacin, piperacillin, ceftazidime, meropenem, amoxicillin/clav, piperacillin, meropenem), vitamins (ADEK, Fer-in-Sol, Polyviflor drops, Aquasol-A, Drisdol, Aquasol-E), pancreatic enzymes (pancrelipase, pancreatin), stool softeners (docusate, casanthranol, polyethylene glycol), GI drugs (omeprazol, ranitidine, metoclopramide), antihistamines (loratadine, cetirizine, fexofenadine), nasal sprays (Vancenase/Vanc AQ, Beconase/BecAQ, sinus rinse), silver sulfadiazine, santyl, urea, hibiclen, Silvadene, Biafine, Sarna, Venelex, Recedo, Rea LO, Luxamend, Bionect, Nuvail, Levicyn, Umecta, Dermasorb XM, Acticoat, Keragelt, Keragel, silver nitrate, mafenide, Sulfamylon, Polysporin, X-Viate, Umecta PD, Uramaxin, remeven, keratolytics, Neosporin, Bacitracin, Neomycin, Polymyxin, Bensal HP, Atrapro, Granulex, Vasolex, zinc paste, calamine, coal tar, ichthammol, pentoxifylline, iloprost, glyceryl trinitrate, calcium antagonists, corticosteroids, psoralen, phenytoin, retinoids, analgesics, colchicine, antiplatelets (aspirin), vasoconstrictors (nicotine, cocaine, adrenaline).

A compound of a fatty acid, such as an odd chain fatty acid, disclosed herein can be used in combination with one or more medical devices, medical treatments, or surgical treatments, e.g., surgical treatments for obesity (e.g., bariatric surgery such as gastric bypass surgery, laparoscopic adjustable gastric banding, biliopancreatic diversion with duodenal switch, gastric sleeve, vagal nerve blockade), catheter-directed thrombolysis, vena cava filter, venous thrombectomy, compression bandaging, vacuum assisted closure, intermittent pneumatic compression device, debridement (sharp, mechanical, autolytic (honey), enzymatic, or biosurgery (maggots)), ultraviolet light therapy, hyperbaric oxygen, radiant heat dressing, ultrasound therapy, laser, hydrotherapy, electrotherapy, electromagnetic therapy, and immunoglobulin replacement therapy.

### Dosing

As will be readily apparent to one skilled in the art, the useful in vivo dosage to be administered and the particular mode of administration will vary depending upon the age, weight, the severity of the condition, and mammalian species treated, the particular forms of the compounds employed, and the specific use for which these compounds are employed. The determination of effective dosage levels, that is the dosage levels necessary to achieve the desired result, can be accomplished by one skilled in the art using routine methods, for example, in vivo studies. Reference may be made to, for example, “Estimating the Maximum Safe Starting Dose in Initial Clinical Trials for Therapeutics in Adult Healthy Volunteers,” U.S. Food and Drug Administration, July 2005.

In some embodiments, a method provided herein may comprise administering a therapeutically effective amount of a composition provided herein. In some embodiments, a therapeutically effective amount may be determined by reference to the modulation of a marker of a condition associated with mood disorders or pain. In some embodiments, a therapeutically effective amount may be determined by reference to the modulation of a symptom of a condition provided herein. In still other embodiments, reference may be made to established guidelines for the conditions described herein, including, but not limited to, guidelines for the treatment of a condition provided herein including mood disorders or pain.

The dosage may vary broadly, depending upon the desired effects and the therapeutic indication, such as marker values. Alternatively, dosages may be based and calculated upon the surface area or weight of the patient, as understood by those of skill in the art. The exact dosage will be determined on a case-by-case basis, or, in some cases, will be left to the informed discretion of the subject. The daily dosage regimen for an adult human patient may be, for example, an oral dose of a fatty acid, such as an odd chain fatty acid or a very long even chain fatty acid, or a salt or derivative thereof, or a mixture of a plurality of fatty acids, or a salt or derivative thereof, from about 0.01 mg to about 10000 mg, from about 1 mg to about 5000 mg, from about 5 mg to about 2000 mg, from about 10 mg to about 1000 mg, or from about 50 mg to about 500 mg. A single dose may include a fatty acid, or a salt or derivative thereof, in about 0.01 mg, about 0.1 mg, about 1 mg, about 5 mg, about 10 mg, about 20 mg, about 50 mg, about 100 mg, about 200 mg, about 300 mg, about 400 mg, about 500 mg, about 600 mg, about 800 mg, about 900 mg, about 1000 mg, about 2000 mg, about 5000 mg, or more. The dosage may be adjusted according to the body mass of the subject, for example, the dosage may be about 0.001 mg/kg, about 0.01 mg/kg, about 0.1 mg/kg, about 0.2 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about mg/kg, about 30 mg/kg, or higher. In some embodiments, the effective amount of the C15:0 fatty acid or pharmaceutically acceptable salt thereof in a pharmaceutical composition is from to 20 mg/kg body weight. The dosage may be a single one or a series of two or more given in the course of one or more days, as is appropriate for the individual subject. In some embodiments, the compounds will be administered for a period of continuous therapy, for example for about a week or more (e.g., one week, two weeks, three weeks, four weeks, five weeks, six weeks, seven weeks, eight weeks, or more), for several weeks, for about a month or more (e.g., one month, two months, three months, four months, five months, six months, seven months, eight months, nine months, ten months, eleven months, twelve months, or more), for about a year or more, or for a plurality of years. In some embodiments, a fatty acid, such as an odd chain fatty acid or a very long even chain fatty acid, or a salt or derivative thereof, can be administered or ingested one time per day, two times per day, three times per day, or more.

As will be understood by those of skill in the art, in certain situations it may be necessary to administer the compounds disclosed herein in amounts that exceed the above-stated, preferred dosage range in order to effectively treat a subject.

Unit dosage forms can also be provided, e.g., individual packages with a premeasured amount of the composition, configured for administration on a predetermined schedule. Unit dosage forms configured for administration one to three times a day are preferred; however, in certain embodiments it may be desirable to configure the unit dosage form for administration more than three times a day, or less than one time per day.

Dosage amount and interval may be adjusted to the individual subject to provide plasma levels of the active moiety which are sufficient to maintain predetermined parameters, indicators, or marker values, or minimal effective concentration (MEC). Dosages necessary to achieve the desired result will depend on individual characteristics and route of administration. However, assays, for example, HPLC assays or bioassays, may be used to determine serum concentrations.

In some embodiments, the compounds and methods provided herein may be used in conjunction with devices and methods of using devices, for example, as provided in U.S. Pat. No. 7,651,845; U.S. Pat. No. 8,251,904; U.S. Pat. No. 8,251,904; U.S. Pat. No. 4,985,015; U.S. Pat. No. 8,827,957; U.S. Pat. No. 4,252,159; U.S. Pat. No. 5,318,521; U.S. Pat. No. 4,718,430; U.S. Pat. No. 9,713,600, U.S. Pat. No. 9,707,199, U.S. Pat. No. 9,687,461, U.S. Pat. No. 9,662,306, U.S. Pat. No. 9,561,206, U.S. Publ. No. 2011/0190702; U.S. Publ. No. 2017/0266144, U.S. Publ. No. 2016/0324814, U.S. Publ. No. 2016/0195559, U.S. Publ. No. 2016/0195558, U.S. Publ. No. 2016/0193172, 2 U.S. Publ. No. 2016/0193171, U.S. Publ. No. 2016/0193170, WO 2016/111843, DE 2615061; and in conjunction with diagnostic devices, for example, as provided in U.S. Publ. No. 2012/0072236.

### Diagnosis and Monitoring

Provided herein are methods for boosting and/or enhancing mood, lowering anxiety and/or pain, treating depression, treating major depressive disorder, or treating seasonal affective disorder.

In some embodiments, the method of diagnosis or monitoring may comprise the step of measuring a percentage of a fatty acid, such as an odd chain fatty acid or a very long even chain fatty acid, in a bodily fluid. In some embodiments, the method of diagnosis or monitoring may comprise the step of measuring a marker of a condition provided herein, including conditions associated with mood disorders or pain, in a subject. In some embodiments, the method of diagnosis or monitoring may comprise the step of measuring a marker of a condition associated with mood disorders or pain. In some embodiments, a correlation between one marker and another may prove instructive. In some embodiments, a condition associated with mood disorders or pain may be diagnosed by reference to a threshold level of erythrocyte sedimentation rate, for example, or serum odd chain fatty acid or serum very long even chain fatty acid. In some embodiments, a condition related to mood disorders or pain provided herein may be diagnosed by reference to a threshold level of a marker of the condition, for example, serum odd chain fatty acid percentage, serum concentration of an odd chain fatty acid, serum total odd chain fatty acid, serum very long even chain fatty acid, serum total very long even chain fatty acids, or a ratio between two serum fatty acids. For example, the threshold may be determined by reference to a symptom or marker of a condition associated with mood disorders or pain.

The percentage of a fatty acid, such as an odd chain fatty acid or a very long even chain fatty acid, or a marker of a condition associated with mood disorders or pain, in a subject may be monitored by any means. Samples for analysis may be derived any fluid or tissue of the subject. For example, from serum, plasma, erythrocyte membranes, urine, and feces.

### EXAMPLES

EXAMPLE 1

Compositions including pentadecanoic acid and salts and derivatives thereof, and methods for mood disorder and chronic pain treatment and prophylaxis are provided, including compositions and methods for boosting and enhancing mood, lowering anxiety and chronic pain, and treating depression, major depressive disorder, and seasonal affective disorder. Pentadecanoic acid has these activities in humans at daily oral doses ranging from 1 mg/day or less to 200 mg/day or more per day, e.g., 1.0 mg/day to 5.0 mg/day, 5 mg/day to 50 mg/day, 20 mg/day to 200 mg/day, etc., as described herein.

### Methods

This study assessed potential pharmacologic targets of pentadecanoic acid. Specifically, this study examined agonist and antagonist activities of synthetic pentadecanoic acid across 78 assays using SAFETYscan E/IC50 ELECT (DiscoverX/Eurofins, Fremont, California). Briefly, a variety of standardized and optimized functional assays were used to assess pentadecanoic acid's pharmacologic targets, including G protein coupled receptors (ADORA2A, ADRA1A, ADRA2A, ADRB1, ADRB2, CB1, CB2, CCK1, D1, D2S, ETA, H1, H2, M1 M2, M3, OPRD1, OPRK1, OPRM1, 5HTR1A, 5HTR1B, 5HTR2A, 5HTR2B, AVPR1A), kinases (LCK, INSR, VEGFR2, ROCK1), transporters (DAT, NET, SERT), ion channels (GABAA, 5-HT3, CA1.2, HERG, KVLQT1/MINK, NA1.5, NMDAR1/2B, NACHR), nuclear receptors (AR, GR), and non-kinase enzymes (COX1, COX2, ACHE, MAOA, PDE3A, PDE4D2). Ten-point concentration curves for both agonist (EC50) and antagonist (IC50) activity were established for each target. Maximum activity (%) was also determined based on comparisons with internal positive controls, which were assigned 100% activity. The positive control for both CB1 and CB2 receptor agonist activity was CP 55940 (EC50=0.06 nm).

### Results

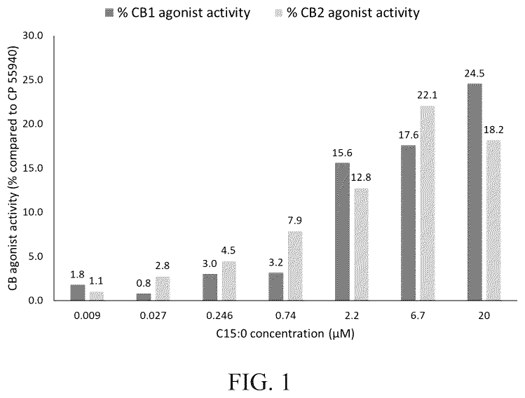

Pentadecanoic acid had dual, partial CB1- and CB2-agonist activity between 2.2 and 20 μM (CB1 =24.5% agonist activity at 20 μM, CB2=18.2% agonist activity at 20 μM). FIG. 1 shows targeted CB1 and CB2 agonist activity of pentadecanoic acid at increasing concentrations.

This study supports that pentadecanoic acid is a dual, partial CB1/CB2 agonist. Based on these activities, pentadecanoic acid may be used at daily doses to achieve at least 2.2 μM in the body to boost and/or enhance mood, lower anxiety and pain, and treat depression, major depressive disorder, and seasonal affective disorder.

EXAMPLE 2

### Methods

This study assessed the human cell phenotypic profile of pentadecanoic acid in order to identify mechanistic similarities with known active compounds.

The Diversity PLUS BioMAP panel allows test agent characterization in an unbiased way across a broad set of systems modeling various human disease states. These systems are designed to model complex human tissue and disease biology of the vasculature, skin, lung, and inflammatory tissues. Quantitative measurements of 148 biomarker activities across this broad panel, along with comparative analysis of biological activities from known bioactive agents, were used to predict and compare the efficacy and function of pentadecanoic acid at eight concentrations ranging from 0.74 to 50 μM.

Each test agent generated a signature BioMAP profile that is created from the changes in protein biomarker readouts within individual system environments. Biomarker readouts (7-17 per system) were selected for therapeutic and biological relevance, were predictive for disease outcomes or specific drug effects and were validated using agents with known mechanism of action (MoA). Each readout was measured quantitatively by immune-based methods that detect protein (e.g., ELISA) or functional assays that measure proliferation and viability. BioMAP readouts were diverse and include cell surface receptors, cytokines, chemokines, matrix molecules and enzymes. In total, the Diversity PLUS panel contained 148 biomarker readouts that capture biological changes that occur within the physiological context of the particular BioMAP system. Specific BioMAP activities have been correlated to in vivo biology, and multiparameter BioMAP profiles have been used to distinguish compounds based on MoA and target selectivity across diverse physiological systems.

Activated BioMAP systems were incubated with each compound for 24 to 72 hours. Protein-based biomarkers from activated cell systems were measured and compared with non-treated control systems. Biomarker activities were noted as ‘significant’ when at least one compound concentration was outside of the significance envelope and had an effect size >20% (log10 ratio)>0.1.

Similarity search analyses were conducted to identify top matches between pentadecanoic acid's cell-based phenotypic profile and other biochemicals in the BioMAP database. The BioMAP database included over 4,500 reference test agents including biologics, approved drugs and experimental chemical compounds. Briefly, a Pearson's correlation coefficient (r) was first generated to measure the linear association between two profiles that was based on the similarity in the direction and magnitude of the relationship. Since the Pearson's correlation can be influenced by the magnitude of any biomarker activity, a per-system weighted average Tanimoto metric was used as a filter to account for underrepresentation of less robust systems. The Tanimoto metric does not consider the amplitude of biomarker activity, but it addresses whether the identity and number of readouts are in common on a weighted, per system basis. Annotated profiles were identified as being mechanistically similar if r≥0.7 and the readout for both profiles was outside of the significance envelope with an effect size >20% (|log₁₀ ratio|>0.1) in the same direction.

### Results

In an unsupervised search for mathematically similar compound profiles from the BioMAP Reference Database, pentadecanoic acid at 1.9 μM was similar to CP 55,940 (1.1 μM) (Pearson's correlation coefficient, r=0.753), and pentadecanoic acid at 20 μM was most similar to buproprion HC1 (30 μM) (Pearson's correlation coefficient, r=0.823). The Pearson's correlation coefficients were above the determined threshold of r=0.7, indicating pentadecanoic acid has mechanistically relevant similarity to both CP 55,940 and buproprion HC1.

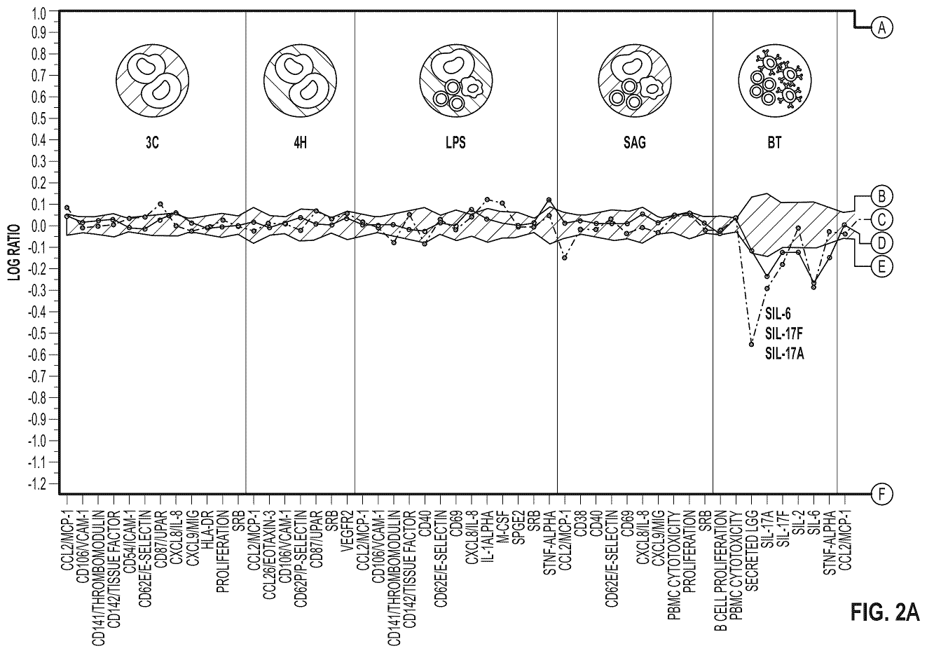

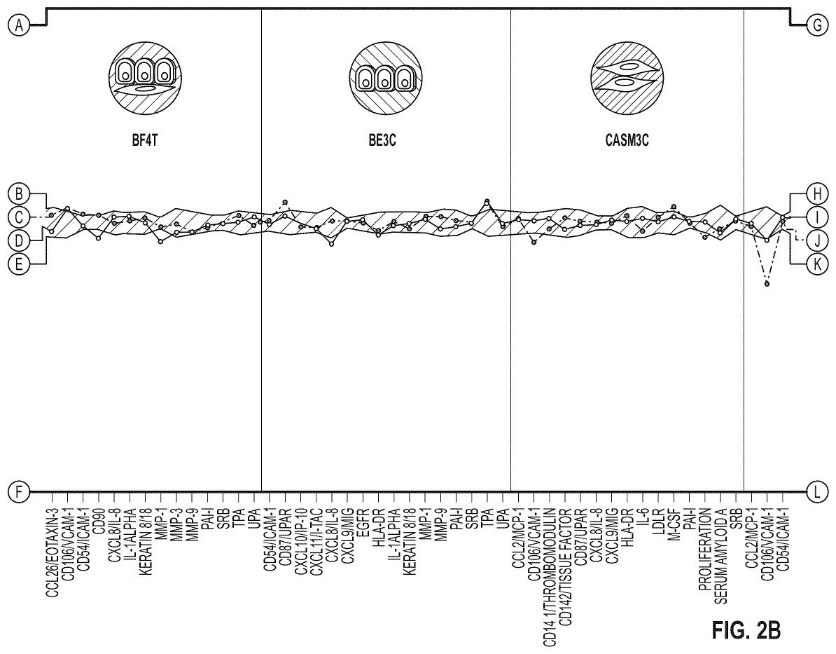

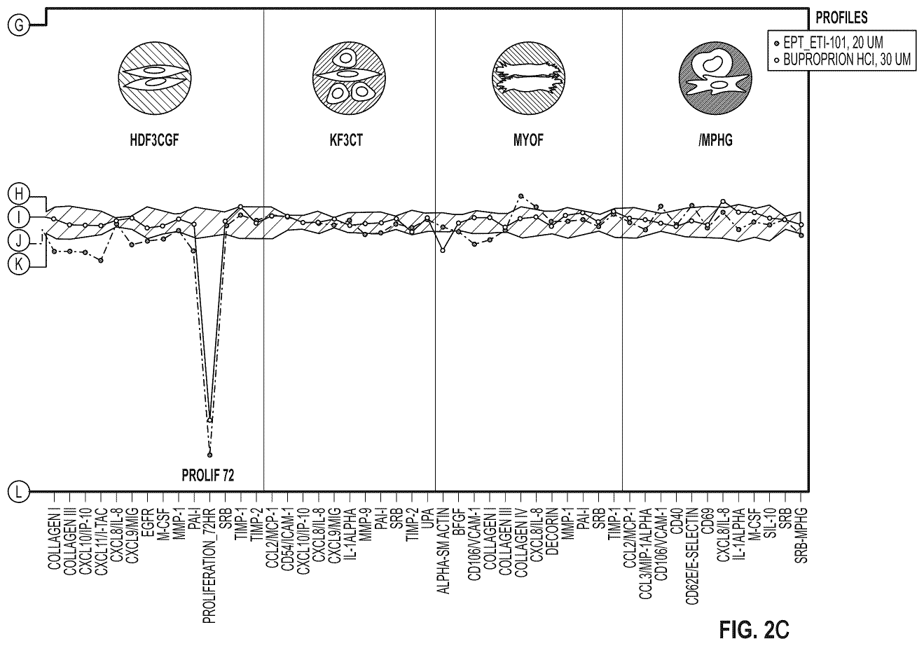

CP 55,940 is a full CB1 and CB2 receptor agonist and synthetic cannabinoid that mimics the effects of naturally occurring tetrahydrocannabinol. Buproprion HC1 is a norepinephrine dopamine reuptake inhibitor, or NDRI, approved by the FDA for use as an antidepressant and smoking cessation aid. As seen in FIGS. 2A-C, the phenotypic profiles of pentadecanoic acid (20 μM) and buproprion HC1 are similar.

In summary, this study demonstrates direct mechanistic similarities between pentadecanoic acid and a CB1/CB2 agonist, as well as an NDRI inhibitor, further supporting use of pentadecanoic acid to achieve pentadecanoic acid concentrations of at least 1.9 uM to boost and enhance mood, lower anxiety and chronic pain, and treat depression, major depressive disorder, and seasonal affective disorder.

Provided in FIGS. 2A-C are Top Database Search Results for pentadecanoic acid (20 μM) is buproprion hydrochloride (30 μM). Overlay of the top similarity match from an unsupervised search of the BioMAP Reference Database of >4,500 agents with pentadecanoic acid (SRP ETI-101-AVA). Common biomarker readouts are annotated when the readout for both profiles is outside of the significance envelope with an effect size >20% (|log10ratio|>0.1) in the same direction. Similarity search results are filtered and ranked as described in Appendix A. Profiles are identified as having mechanistically relevant similarity if the Pearson's correlation coefficient is ≥0.7.

EXAMPLE 3

An odd-chain saturated fatty acid dietary supplement, containing an encapsulated pure (>98%) free fatty acid C15:0 powder and called fatty15™, was available as a consumer health product. Recommended dosing is a single, 100 mg capsule taken orally once a day. Based on our studies demonstrating C15:0 as a partial CB-1 agonist and with a human cell phenotypic profile similar to that of bupropion, we hypothesized that daily oral C15:0 supplementation would lower stress and enhance mood.

6-Week Survey. Approximately six weeks following receipt of the C15:0 supplement, fattyl5, customers were provided a voluntary electronic questionnaire to evaluate potential near-term benefits, including the questions: “Overall how much would you agree with the following statement: Since starting fatty15, I have been feeling more calm and/or less stressed.” and “Overall how much would you agree with the following statement: Since starting fatty15, I generally feel happier and/or not as bothered by things that used to upset me.”

Survey responders were asked to select a number between 1 and 10, in which 1=Strongly disagree and 10=Strongly agree. A response of 7 or greater was considered an agreement with the statement. Survey responders were also asked if fatty15 had become a part of their daily health routine.

A total of 135 people responded to the 6-week survey. Almost all (97%) reported that fattyl5 had become a part of their daily health routine. After 6 weeks of daily oral supplementation of 100 mg C15:0, 41% of 135 customers reported feeling calmer and/or less stressed; and 37% reported feeling generally happier and/or not as bothered by things that used to upset them.

These data support that daily oral supplementation with an odd-chain saturated fatty acid, specifically C15:0 at 100 mg/day for 6 weeks can support a calmer, less stressed, and happier mood.

Example 2

An odd-chain saturated fatty acid dietary supplement, containing an encapsulated pure (>98%) free fatty acid C15:0 powder and called fatty15™, is available as a consumer health product. Recommended dosing is a single, 100 mg capsule taken orally once a day. Customers were provided opportunities to share their experience, including through voluntary surveys and customer rating programs.

Table 2 provides a summary of comments provided by fatty15™ customers related to stress and mood.

Examples of Customer Testimonials

I definitely feel that things that normally bothered me & just my general overall mood is less
stressed/more relaxed
I do feel better and my mood is improved.
I think perhaps I am calmer.
I think I've seen a slight improvement in stress management
Definitely less stressed!
Very impressed with the sustained improvements in stress.
Less stress and in better mood.
Since the pandemic started in March, I became very stressed and found myself snacking
frequently, gaining weight, and losing sleep. I have noticed several changes in my habits since
starting fatty 15 a month ago. Four most notable effects that I have experienced are (1) my
lessened desire to snack in between meals and (2) losing weight and ease in maintaining my
lower weight (the lowest I've been in 3 years!!)
My productivity has increased and my stress levels have decreased
I feel less stressed
Happier, less stressed frame of mind
I feel really great. I'm happier--more content & calm overall and am sleeping better.
Overall improved calm
Makes me feel calmer.
I really feel my stress levels have improved.

These testimonials support that daily oral supplementation with an odd-chain saturated fatty acid, specifically C15:0 at 100 mg/day, can lower stress and calm and enhance mood.

### EXEMPLARY EMBODIMENTS

Method 1: A method of boosting and/or enhancing mood, lowering anxiety and/or pain, treating depression, treating major depressive disorder, or treating seasonal affective disorder, comprising: administering, to a patient in need thereof, an effective amount of a C15:0 fatty acid or pharmaceutically acceptable salt thereof in a pharmaceutical composition, a dietary supplement, or a food.

Method 2: Method 1, wherein administering is administering as a component of a food.

Method 3: Method 2, wherein administering is administering as a dietary supplement.

Method 4: Method 1, for boosting and/or enhancing mood.

Method 5: Method 1, for lowering pain.

Method 6: Method 1, for lowering anxiety.

Method 7: Method 1, for treating depression.

Method 8: Method 1, for treating major depressive disorder.

Method 9: Method 1, for treating seasonal affective disorder.

Method 10: Any one of Methods 1 through 9, wherein a serum, plasma, or a red blood cell membrane concentration of the C15:0 fatty acid is increased to a concentration greater than 2.2 μM and less than 30 μM.

Method 11: Any one of Methods 1 through 10, wherein the C15:0 fatty acid is pentadecanoic acid.

Method 12: Any one of Methods 1 through 11, wherein the C15:0 fatty acid or pharmaceutically acceptable salt thereof is provided as a pharmaceutical composition in a unit dosage form comprising the C15:0 fatty acid or pharmaceutically acceptable salt thereof and a pharmaceutically acceptable carrier.

Method 13: Method 12, wherein the unit dosage form comprises from 0.01 mg to 10000 mg of the C15:0 fatty acid or pharmaceutically acceptable salt thereof.

Method 14: Any one of Methods 12 through 13, wherein the pharmaceutical composition is substantially free from even chain saturated fatty acids.

Method 15: Any one of Methods 12 through 14, wherein the pharmaceutical composition is substantially free from polyunsaturated fatty acids.

Method 16: Any one of Methods 12 through 15, wherein the C15:0 fatty acid or pharmaceutically acceptable salt thereof is administered to the patient once per day.

Method 17: Any one of Methods 1 through 16, wherein the patient is a human.

Method 18: Any one of Methods 1 through 16, wherein the patient is a mammal.

Method 19: Any one of Methods 1 through 16, wherein the patient is a domesticated animal.

Method 20: Method 19, wherein the domesticated animal is a dog or a cat.

Method 21: Method 9, wherein the domesticated animal is a cow, a pig, a sheep, a goat, a horse, a turkey, a duck, or a chicken.

Method 22: Any one of Methods 1 through 21, wherein from 20 to 200 mg/kg body weight of the C15:0 fatty acid or pharmaceutically acceptable salt thereof per day is administered to the patient.

Composition 23: A composition for boosting and/or enhancing mood, lowering anxiety and/or pain, treating depression, treating major depressive disorder, or treating seasonal affective disorder, comprising: a C15:0 fatty acid or pharmaceutically acceptable salt thereof; and a pharmaceutically acceptable carrier.

Composition 24: Composition 23, which is a pharmaceutical composition in unit dosage form.

Composition 25: Composition 23, which is a dietary supplement.

Composition 26: Composition 23, wherein the dietary supplement is in unit dosage form.

Composition 27: Composition 23, wherein the dietary supplement is in a form adapted to be combined with or added to a food, beverage, or other comestible.

Composition 28: Composition 23, wherein the composition is a food or other comestible.

Composition 29: Any one of Compositions 23 through 28, or boosting and/or enhancing mood.

Composition 30: Composition 29, for boosting mood.

Composition 31: Composition 29, for enhancing mood.

Composition 32: Any one of Compositions 23 through 28, for lowering anxiety.

Composition 33: Any one of Compositions 23 through 28, for lowering pain.

Composition 34: Any one of Compositions 23 through 28, for treating depression.

Composition 35: Any one of Compositions 23 through 28, for treating major depressive disorder.

Composition 36: Any one of Compositions 23 through 28, for treating seasonal affective disorder.

Composition 37: Any one of Compositions 23 through 36, adapted to increase a serum, plasma, or a red blood cell membrane concentration of the C15:0 fatty acid or pharmaceutically acceptable salt thereof to a concentration greater than 2.2 μM and less than 30 μM.

Composition 38: Any one of Compositions 23 through 37, wherein the C15:0 fatty acid or pharmaceutically acceptable salt thereof is pentadecanoic acid.

Composition 39: Composition 26, wherein the unit dosage form comprises from 0.01 mg to 10000 mg of the C15:0 fatty acid or pharmaceutically acceptable salt thereof.

Composition 40: Any one of Compositions 23 through 39, wherein the pharmaceutical composition is substantially free from even chain saturated fatty acids.

Composition 41: Any one of Compositions 23 through 40, wherein the pharmaceutical composition is substantially free from polyunsaturated fatty acids.

Composition 42: Any one of Compositions 23 through 41, adapted for administration of 0.2 to 20 mg/kg body weight of the C15:0 fatty acid or pharmaceutically acceptable salt thereof per day to a patient in need thereof.

Composition 43: Composition 26, wherein the unit dosage form is adapted for administration to the patient once per day.

Composition 44: Any one of Compositions 23 through 43, wherein the patient is a human.

Composition 45: Any one of Compositions 23 through 43, wherein the patient is a mammal.

Composition 46: Any one of Compositions 23 through 43, wherein the patient is a domesticated animal.

Composition 47: Composition the domesticated animal is a dog or a cat.

Composition 48: Composition the domesticated animal is a cow, a pig, a sheep, a goat, a horse, a turkey, a duck, or a chicken.

Use 49: Use of a composition for boosting and/or enhancing mood, lowering anxiety and/or pain, treating depression, treating major depressive disorder, or treating seasonal affective disorder, the composition comprising: C15:0 fatty acid or pharmaceutically acceptable salt thereof; and a pharmaceutically acceptable carrier.

Use 50: Use 49, for boosting and/or enhancing mood.

Use 51: Use 50, for boosting mood.

Use 52: Use 50, for enhancing mood.

Use 53: Use 49, for lowering anxiety.

Use 54: Use 49, for lowering pain.

Use 55: Use 49, for treating depression.

Use 56: Use 49, for treating major depressive disorder.

Use 57: Use 49, for treating seasonal affective disorder.

Use 58: Any one of Uses 49 through 57, wherein a serum, plasma, or a red blood cell membrane concentration of the C15:0 fatty acid is increased to a concentration greater than 2.2 I′M and less than 30 μM.

Use 59: Any one of Uses 49 through 58, wherein the C15:0 fatty acid is pentadecanoic acid.

Use 60: Any one of Uses 49 through 59, wherein the C15:0 fatty acid or pharmaceutically acceptable salt thereof is provided as a pharmaceutical composition in a unit dosage form comprising the C15:0 fatty acid or pharmaceutically acceptable salt thereof and a pharmaceutically acceptable carrier.

Use 61: Use 60, wherein the unit dosage form comprises from 0.01 mg to 10000 mg of the C15:0 fatty acid or pharmaceutically acceptable salt thereof.

Use 62: Any one of Uses 60 through 61, wherein the pharmaceutical composition is substantially free from even chain saturated fatty acids.

Use 63: Any one of Uses 60 through 62, wherein the pharmaceutical composition is substantially free from polyunsaturated fatty acids.

Use 64: Any one of Uses 60 through 63, wherein the C15:0 fatty acid or pharmaceutically acceptable salt thereof is administered to the patient once per day.

Use 65: Any one of Uses 49 through 64, wherein the patient is a human.

Use 66: Any one of Uses 49 through 64, wherein the patient is a mammal. Use 67: Any one of Uses 49 through 64, wherein the patient is a domesticated

animal.

Use 68: Use 67, wherein the domesticated animal is a dog or a cat.

Use 69: Use 67, wherein the domesticated animal is a cow, a pig, a sheep, a goat, a horse, a turkey, a duck, or a chicken.

Use 70: Any one of Uses 49 through 69, wherein from 0.2 to 20 mg/kg body weight of the C15:0 fatty acid or pharmaceutically acceptable salt thereof is administered to the patient per day.

Use 71: Any one of Uses 49 through 70, wherein the C15:0 fatty acid or pharmaceutically acceptable salt thereof is administered as a component of a food.

The above description presents the best mode contemplated for carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above that are fully equivalent. Consequently, this invention is not limited to the particular embodiments disclosed. On the contrary, this invention covers all modifications and alternate constructions coming within the spirit and scope of the invention as generally expressed by the following claims, which particularly point out and distinctly claim the subject matter of the invention. While the disclosure has been illustrated and described in detail in the drawings and foregoing description, such illustration and description are to be considered illustrative or exemplary and not restrictive.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ including but not limited to,' or the like; the term ‘comprising’ as used herein is synonymous with ‘including,’ containing,' or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred’, ‘desired’, or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article ‘a’ or ‘an’ does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases ‘at least one’ and “one or more” to introduce claim recitations. However, the use of such phrases should not be construed to imply that the introduction of a claim recitation by the indefinite articles ‘a’ or ‘an’ limits any particular claim containing such introduced claim recitation to embodiments containing only one such recitation, even when the same claim includes the introductory phrases ‘one or more’ or ‘at least one’ and indefinite articles such as ‘a’ or ‘an’ (e.g., ‘a’ and/or ‘an’ should typically be interpreted to mean ‘at least one’ or ‘one or more’); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of ‘two recitations,’ without other modifiers, typically means at least two recitations, or two or more recitations). Furthermore, in those instances where a convention analogous to ‘at least one of A, B, and C, etc.’ is used, in general such a construction is intended in the sense one having skill in the art would understand the convention (e.g., ‘a system having at least one of A, B, and C’ would include but not be limited to systems that have A alone, B alone, C alone, A and B together, A and C together, B and C together, and/or A, B, and C together, etc.). In those instances where a convention analogous to ‘at least one of A, B, or C, etc.’ is used, in general such a construction is intended in the sense one having skill in the art would understand the convention (e.g., ‘a system having at least one of A, B, or C’ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase ‘A or B’ will be understood to include the possibilities of ‘A’ or ‘B’ or ‘A and B.’

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

What is claimed is:

1. A composition for boosting and/or enhancing mood, lowering anxiety and/or pain, treating depression, treating major depressive disorder, or treating seasonal affective disorder, comprising:
C15:0 fatty acid or pharmaceutically acceptable salt thereof; and
a pharmaceutically acceptable carrier.

2. The composition of claim 1, wherein the composition is in the form of a dietary supplement.

3. The composition of claim 2, wherein the dietary supplement is in unit dosage form.

4. The composition of claim 2, wherein the dietary supplement is in a form adapted to be combined with or added to a food, beverage, or other comestible.

5. A method of boosting and/or enhancing mood, lowering anxiety and/or pain, treating depression, treating major depressive disorder, or treating seasonal affective disorder, comprising:
administering, to a patient in need thereof, an effective amount of a C15:0 fatty acid or pharmaceutically acceptable salt thereof in a pharmaceutical composition, a dietary supplement, or a food.

6. The method of claim 5, wherein administering is administering as a component of a food.

7. The method of claim 5, wherein administering is administering as a dietary supplement.

8. The method of claim 5, wherein a serum, plasma, or a red blood cell membrane concentration of the C15:0 fatty acid is increased to a concentration greater than 2.2 μM and less than 30 μM.

9. The method of claim 5, wherein the C15:0 fatty acid is pentadecanoic acid.

10. hod of claim 5, wherein the C15:0 fatty acid or pharmaceutically acceptable salt thereof is provided as a pharmaceutical composition in a unit dosage form comprising the C15:0 fatty acid or pharmaceutically acceptable salt thereof and a pharmaceutically acceptable carrier.

11. The method of claim 10, wherein the unit dosage form comprises from 0.01 mg to 10000 mg of the C15:0 fatty acid or pharmaceutically acceptable salt thereof.

12. The method of claim 11, wherein the unit dosage form comprises 100 mg to 200 mg of the C15:0 fatty acid or pharmaceutically acceptable salt thereof.

13. The method of claim 10, wherein the pharmaceutical composition is substantially free from even chain saturated fatty acids.

14. The method of claim 10, wherein the pharmaceutical composition is substantially free from polyunsaturated fatty acids.

10. od of claim 10, wherein the pharmaceutical composition is substantially free from C17:0 fatty acid.

16. The method of claim 10, wherein the C15:0 fatty acid or pharmaceutically acceptable salt thereof is administered to the patient once per day.

17. The method of claim 5, wherein the patient is a human.

18. The method of claim 5, wherein the patient is a domesticated animal.

19. The method of claim 5, wherein the effective amount of the C15:0 fatty acid or pharmaceutically acceptable salt thereof in a pharmaceutical composition is from 0.2 to 20 mg/kg body weight.

19. od of claim 19, wherein the effective amount of the C15:0 fatty acid is about 5 mg/kg body weight.
